(19) United States
(12) Patent Application Publication
(10) Pub. No.: US 2008/0108903 A1
(43) Pub. Date: May 8, 2008

(54) Portable respiration monitoring and feedback system

(76) Inventors: Nir Ben-Oved, Vancouver (CA); Gregory Thomas Sheehan, North Vancouver (CA)

Correspondence Address: Nir Ben-Oved, 639 East 29th Ave, Vancouver, BC, V5V2S1, omitted

(21) Appl. No.: 11/983,161

(22) Filed: Nov. 8, 2007

Related U.S. Application Data
(60) Provisional application No. 60/857,261, filed on Nov. 8, 2006.

(51) Int. Cl. A61B 5/08; A61B 5/113

(52) U.S. Cl. 600/484; 600/534

(57) ABSTRACT

A respiration feedback monitoring and feedback system, and in particular, an apparatus and corresponding method for monitoring and controlling respiration activity of a user that encompasses a respiration monitor sized and configured to be worn by the user. The respiration activity of the user is measured with components including a signal generator and a self-retaining belt which coils and uncoils within housing. Feedback is provided to the user using non-audible or audible signals, such as vibrations of certain duration and repetition or music players. A method for determining appropriate feedback corresponding to the user's respiration activity is also provided. The method includes defining the user's desired respiratory activity and respiration feedback criteria for the determination of the appropriate feedback. The method further includes various user selectable operational variables.

### CROSS REFERENCE TO RELATED PATENT APPLICATION

This application claims the benefit of the filing date of U.S. provisional patent application Ser. No. 857,261, filed Nov. 08, 2006, which is hereby incorporated by reference in its entirety.

### FIELD OF THE INVENTION

This invention relates to the field of physiological monitoring systems and more particularly to a wearable self contained respiration feedback monitoring system.

### STATEMENT REGARDING FEDERALLY SPONSORED RESEARCH OR DEVELOPMENT

Not applicable

### REFERENCE TO SEQUENCE LISTING, A TABLE, OR A COMPUTER PROGRAM LISTING COMPACT DISC APPENDIX

Not applicable

### BACKGROUND OF THE INVENTION

An individual's health and fitness level may be determined by measuring his or her breathing patterns during respiration. In turn, respiration patterns also influence the fitness level and health of the individual. Respiration patterns are typically measured with air bladders or piezoelectric sensors, which use the deformation of the material as it applies to respiration movements. Two components of the measured respiration patterns are respiration rate and respiration depth. Respiration rate is a measure of the number of breaths taken per unit time, typically measured in breaths per minute. Respiration depth is a measure of the extent to which an individual's lungs expand and contract.

Many specific health ailments and fitness problems can be correlated to particular breathing patterns, specifically respiration rate and respiration depth. Studies have shown that certain individuals do not breathe properly when under stress or when concentrating, which in turn leads to health problems. These individuals are usually unaware that while concentrating or under stress, their respiration becomes improper. Fortunately, this improper respiration has discernable patterns. For example, oftentimes such improper respiration may be characterized by breaths that are too shallow or infrequent. It is generally understood that proper breathing is diaphragmatic as opposed to accessory or chest breathing.

Diaphragmatic breathing causes the diaphragm muscle to contract by pulling the bottom of the lungs downward, causing them to fill, while the ribs flare outward to the sides. The chest and abdominal muscles are not used in diaphragmatic breathing. Diaphragmatic breathing aids proper blood circulation by drawing blood back to the heart and also massages and stimulates the organs of the abdominal cavity. The ability for people to self regulate diaphragmatic breathing would be of tremendous benefit to manage stress.

However, many of us lead stress-filled lives, and learn bad breathing habits, using the chest. This creates further tension that leads to physical tightness. The diaphragm of people who predominantly employ chest breathing, which is shallow, will gradually weaken. This weakened condition often causes the person to be more susceptible to various respiratory problems and infections. Chest breathing tends to cause unnecessary tension in the body while, conversely, diaphragmatic breathing tends to eliminate this tension. In fact, many stress-control exercises, such as yoga and the like, emphasize proper diaphragmatic breathing as a form of relaxation, to promote a natural and healthy sleep and to improve general health.

Prior art devices do not emphasize use during daily life activities and are bulky. In addition, these devices burden the user and require medical assistance to support, train and regulate the users breathing. In the prior art, attempts have been made to monitor respiration to a limited extent and to provide a form of feedback to the individual whose respiration is being monitored. Unfortunately, known devices are not conducive for use during an individual's normal daily activities. The devices which measure all of the respiration components are bulky and are usually limited to fixed locations such as clinics, hospitals or sophisticated training centers, placing further constraints and demands upon individuals attempting to improve their respiration patterns and breathing practices. The medical community does not focus on diaphragmatic breathing but rather on symptom specific breathing related patterns. If individuals were self aware of their respiration patterns throughout their daily life, especially when engaged in stressful activities, such as work or driving, this information could help them improve their breathing habits and consequently improve their health. In addition, if the user is able to perform self monitoring without the guidance of a professional, this would allow the user to become self educated in proper diaphragmatic breathing.

One of the disadvantages of prior art devices is their inability to accommodate daily activities. An emphasis is not generally placed on the comfort of the user or the use of a device for daily wear. Another disadvantage is that in prior art devices, users usually do not actively determine the breathing patterns they would like to be reminded of. For instance, U.S. Pat. No. 6,162,183 Hoover teaches a prior art portable respiration feedback monitor based on an onsite located computer controlled system used to program and analyze historical respiration data. The program is configured to compare the users breathing with a predetermined criteria and to track progress. While this apparatus includes several function modes it lacks the participation of the user in choosing personal breathing patterns and professional input to interpret the results and to adapt the criteria accordingly. Another aspect of the prior art devices which makes them inappropriate for daily use is that most of them consist of a belt that is external to the device. This generally makes the device more bulky, difficult and complicated to carry and less user-friendly. For instance, U.S. Pat. No. 4,909,260 to Salem et al. describes a portable respiration monitor. However, Salem's monitor is too bulky and cumbersome to be used in many daily activities, and, as with other prior art devices. In addition the feedback methods in prior art devices are inappropriate for daily use and pose significant disadvantages, for example Salem's feedback mechanism uses visual apparatus to provide feedback, which requires the user to focus attention on the visual apparatus rather than his or her breathing. Salem's respiration monitor requires a sacrifice in lifestyle, wardrobe, and may also potentially embarrass the user by drawing public attention to the visual feedback apparatus If individuals were able to be provided feedback that is discrete in nature, this would allow the user to feel the feedback and make personal adjustments.

There remains, therefore, a continuing need in breathing monitor technology to provide the benefits of proper breathing while functionally and unobtrusively integrating with daily lifestyles.

### BRIEF SUMMARY OF THE INVENTION

The invention overcomes the limitations of the prior art and provides additional benefits for a respiration feedback monitor system. The respiration feedback monitor allows for expanded accessibility under a wide range of activities. The feedback is discrete in nature. In addition, the invention has the additional advantage of a belt that is self retained and self-contained within the apparatus thus it makes the apparatus less bulky and easier to carry around for daily use. In addition, the belt is worn around the user's torso which makes it more adaptable for daily use and emphasizes diaphragmatic breathing. Thus, the invention overcomes the problems and difficulties posed by the prior art systems and provides numerous additional benefits.

An aspect of the invention includes a housing sized and configured to be worn by a user, a belt coiled on rolling rods within the housing, signal generators connected to the rolling rods generate signals corresponding to the coiling and uncoiling movements of the belt on the rolling rods, a spiral spring is connected to the rolling rods and creates a torque to coil the belt on the rolling rods, and an output device configured to transmit a feedback signal perceptible by the user when the output device is activated.

In one embodiment, two rolling rods configured to coil and uncoil a belt extended from both sides of the housing with respect to changes in the abdominal circumference corresponds to respiration of the user. Signal generators generate signals corresponding to the coiling and uncoiling movements of the belt on the rolling rods. The signal generators are connected directly to the rolling rods. Spiral springs are connected to each of the rolling rods and create a torque to coil the belt on the rolling rods. The spiral springs are situated in spiral spring housings. The spiral spring housings are affixed to the housing.

In one embodiment, two rolling rods configured to coil and uncoil a belt extended from both sides of the housing with respect to changes in the abdominal circumference corresponds to respiration of the user. Signal generators generate signals corresponding to the coiling and uncoiling movements of the belt on the rolling rods. The signal generators are connected directly to the rolling rods. Spiral springs are connected to each of the rolling rods and create a torque to coil the belt on the rolling rods. The spiral springs are situated in spiral spring housings. The spiral spring housing can be rotated in relation to the spring axis, thus to control the torque the spring is applying on the rolling rods. The spiral spring housing rotary movement is controlled by a motor. The motor is connected directly to the spring housing.

In one embodiment, two rolling rods configured to coil and uncoil a belt extended from both sides of the housing with respect to changes in the abdominal circumference corresponds to respiration of the user. Signal generators generate signals corresponding to the coiling and uncoiling movements of the belt on the rolling rods. The signal generators are connected directly to the rolling rods. Spiral springs are connected to each of the rolling rods and create a torque to coil the belt on the rolling rods. The spiral springs are situated in spiral spring housings. The spiral spring housing can be rotated in relation to the spring axis, thus to control the torque the spring is applying on the rolling rods. The spiral spring housing rotary movement is controlled by a motor. The motor is connected indirectly to the spring housing with worm gear reduction.

In one embodiment, signal generator generates signals corresponding to the coiling and uncoiling movements of the belt on the rolling rod. The signal generator is connected directly to one of the rolling rods. Rolling member is connected to each of the rolling rods with gears. Spiral spring is connected to the rolling member and creates a torque on the rolling member. The torque is transmitted with the gears to coil the belt on the rolling rods. The spiral spring is situated in spiral spring housing. The spiral spring housing is affixed to the housing.

In one embodiment, signal generator generates signals corresponding to the coiling and uncoiling movements of the belt on the rolling rod. The signal generator is connected directly to one of the rolling rods. Rolling member is connected to each of the rolling rods with gears. Spiral spring is connected to the rolling member and creates a torque on the rolling member. The torque is transmitted with the gears to coil the belt on the rolling rods. The spiral spring is situated in spiral spring housing. The spiral spring housing can be rotated in relation to the spring axis, thus to control the torque the spring is applying on the rolling rods. The spiral spring housing rotary movement is controlled by a motor. The motor is connected directly to the spring housing.

In one embodiment, signal generator generates signals corresponding to the coiling and uncoiling movements of the belt on the rolling rod. The signal generator is connected directly to one of the rolling rods. Rolling member is connected to each of the rolling rods with gears. Spiral spring is connected to the rolling member and creates a torque on the rolling member. The torque is transmitted with the gears to coil the belt on the rolling rods. The spiral spring is situated in spiral spring housing. The spiral spring housing can be rotated in relation to the spring axis, thus to control the torque the spring is applying on the rolling rods. The spiral spring housing rotary movement is controlled by a motor. The motor is connected indirectly to the spring housing with worm gear reduction.

A method is used to determine whether to activate or deactivate a feedback signal. The method includes a reset procedure in which the users determine their desired pattern, i.e. their breathing depth and their breathing rate. The method further includes feedback criteria that determine whether to activate or deactivate a feedback signal considering the respiratory data collected with the reset procedure. The feedback criteria further includes data determined by the user with user controls.

User controls are used to select different respiratory feedback criteria variables, including type of feedback signal, properties of feedback signal, amount of feedbacks, type of respiratory data to consider into feedback criteria, and type of feedback criteria. User controls are also used to determine the belt force the user sense while using the breathing monitor device.

Different types of feedback signals are available of the user. This includes a tactile vibration feedback, increasing belt force feedback, and reducing voice volume feedback.

### BRIEF DESCRIPTION OF THE DRAWINGS

The present invention utilizes a belt in the depicted embodiment, however any other accessory can be used including but not limited a strip, strap, material or fanny pack.

A better understanding of the present invention can be obtained when the following detailed description of the preferred embodiment is considered in conjunction with the following drawings, in which:

FIG. 1 is a front view of one embodiment of the invention in use.

FIG. 1A is a side view of one embodiment of the invention in use.

FIG. 2 is an isometric view of the first embodiment of the invention with two rolling rods for coiling and uncoiling of a belt, two signal generators situated on each rolling rod, and two spiral springs situated on each rolling rod, each spring is situated in spring housing, and the spring housings are affixed to the housing.

FIG. 3 is an isometric view of the second embodiment of the invention with two rolling rods for coiling and uncoiling of a belt, two signal generators situated on each rolling rod, and two spiral springs situated on each rolling rod, each spring is situated in a spring housing that is connected directly to a motor.

FIG. 4 is an isometric view of the third embodiment of the invention with two rolling rods for coiling and uncoiling of a belt, two signal generators situated on each rolling rod, and two spiral springs situated on each rolling rod, each spring is situated in a spring housing with the spring housing connected indirectly to a motor via a worm gear reduction.

FIG. 5 is an isometric view of the fourth embodiment of the invention with two rolling rods for coiling and uncoiling of a belt, one signal generator situated on one of the rolling rods, a rolling member connected to both rolling rods with gears, and a spiral spring situated on the rolling member, the spring is situated in a spring housing that is affixed to a housing.

FIG. 6 is an isometric view of the fifth embodiment of the invention with two rolling rods for coiling and uncoiling of a belt, one signal generator situated on one of the rolling rods, a rolling member connected to both rolling rods with gears, and a spiral spring situated on the rolling member, the spring is situated in a spring housing and connected directly to a motor.

FIG. 7 is an isometric view of the sixth embodiment of the invention with two rolling rods for coiling and uncoiling of a belt, one signal generator situated on one of the rolling rods, a rolling member connected to both rolling rods with gears, and a spiral spring situated on the rolling member, the spring is situated in a spring housing that is connected indirectly to a motor via a worm gear reduction.

FIG. 8 is a block diagram of electronic components of the invention embodiments shown in FIG. 1 through FIG. 7.

FIG. 9 is a plot of the measured rotational movement (alpha) versus time, illustrating respiration characteristics measured by the embodiment of the invention shown in FIG. 1 through FIG. 8.

FIG. 10 is a top view of a vibration monitor that is utilized in the embodiments of the invention described in FIG. 1 through FIG. 8

### DETAILED DESCRIPTION OF THE INVENTION

A portion of the disclosure of this patent document may contain material which is subject to copyright protection. The copyright owner has no objection to the facsimile reproduction by anyone of the patent document or the patent disclosure as it appears, in the Patent and Trademark Office file or records, but otherwise reserves all copyright rights whatsoever.

Several embodiments of a respiration monitor, and in particular, an apparatus and corresponding method for a respiration monitoring and feedback system are described in detail below. In the following description, numerous specific details are provided, such as specific configuration of the apparatus, circuit components, ways of wearing the respiration monitor, respiration criteria used for feedback, etc., to provide a thorough understanding of the embodiments of the invention. One skilled in the relevant art, however, will appreciate and recognize that the invention can be used with or without one or more of the specific details or with other components, processes, configurations, and operations.

In some instances, in the description below, well-known structures, components or operations are not shown or described in detail to avoid obscuring the description of the embodiments. For example, all of the electrical and electronics used in the described embodiments of the invention are 19 of types well-known in the art such that one skilled in the art would be able to use such circuits in the described combination without further instructions. The internal details of these particular circuits are neither part of, nor critical to, the invention and therefore not provided.

The invention solves various problems of prior art respiration monitors. Prior art respiration feedback monitors are burdensome to use and provide insufficient feedback. The invention is lightweight and compact, and, for example, can be worn throughout the day and night and during common activities without sacrificing lifestyle or wardrobe. Also, the invention is self contained and simple to operate, which promotes ease of use. Furthermore, the invention provides a discreet feedback mechanism, such as tactile feedback or increasing force feedback, allowing the use of the respiration monitor in most situations and environments common in everyday life. The discreet feedback mechanism does not require the user's continuous attention. All these features and advantages of the invention stand in sharp contrast to prior art systems, which are limited to certain locations, environments, or activities and also do not monitor full respiration patterns nor provide direct feedback. Given the ease of use and the great range of locations and environments in which the invention can be used, users, can benefit from the respiration feedback mechanism in a greater variety of their daily or nightly activities by taking measures to correct or improve their respiration patterns and consequentially their health condition and fitness level.

In order to monitor respiration patterns continually, the user should ideally wear a respiration monitor that does not significantly detract from his or her normal activities throughout the day, nor significantly impacts any other aspect of his or her lifestyle.

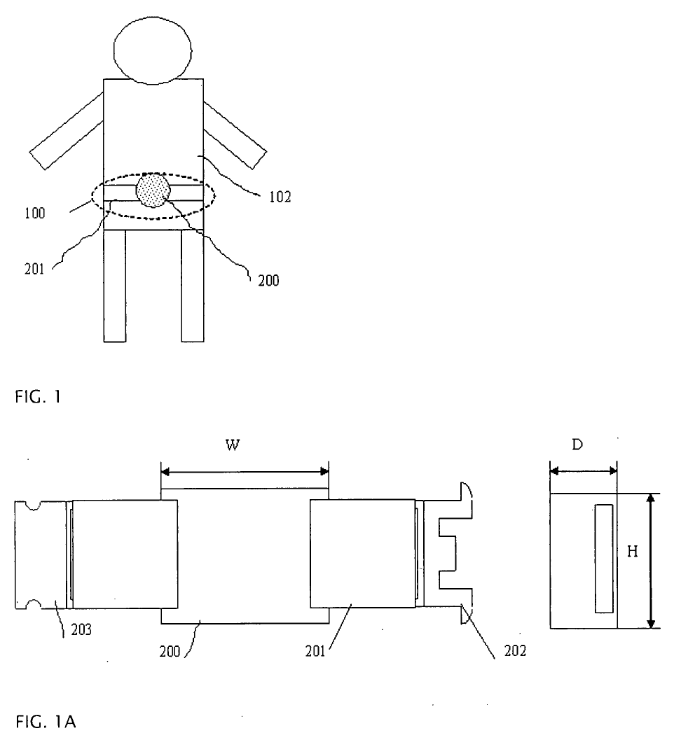

Referring to FIG. 1 and FIG. 1A, in one embodiment of the invention, a respiration monitor 100 includes a housing 200, a belt 201, and belt buckles 202 and 203. The belt 201, shown in its extended position, is anchored within the housing 200 where it is coiled and from the sides of which it can partially or fully extend depending on whether the respiration monitor 100 is being stored or worn, and on the girth of a user 102. The belt 201 is wrapped around a torso of a user 102 and secured by fasteners 202 and 203 which allow the belt to be fit unobtrusively across the torso. The location of the respiration monitor 100 on the user's body is generally at or around the user's diaphragmatic region such that the belt 201 mirrors the expansions and contractions of the user's diaphragm (not shown).

Still referring to FIG. 1 and FIG. 1A, the housing 200 is generally small, such as about 4 inches or smaller in height (H on FIG. 1A) and about 2 inches or smaller in width (W on FIG. 1A) and about 1.5 inches or smaller in depth (D on FIG. 1A). The small size of the housing 200 greatly contributes to the wearability of the respiration feedback monitor 100 and enables it to be worn in a variety of activity and implemented on or within a variety of articles of clothing. Furthermore, one skilled in the art will appreciate and recognize that the ways of wearing the respiration monitor 100 and the body regions where the respiration monitor may be worn are not limited in any way by the description herein, but will depend on requirements of a particular application.

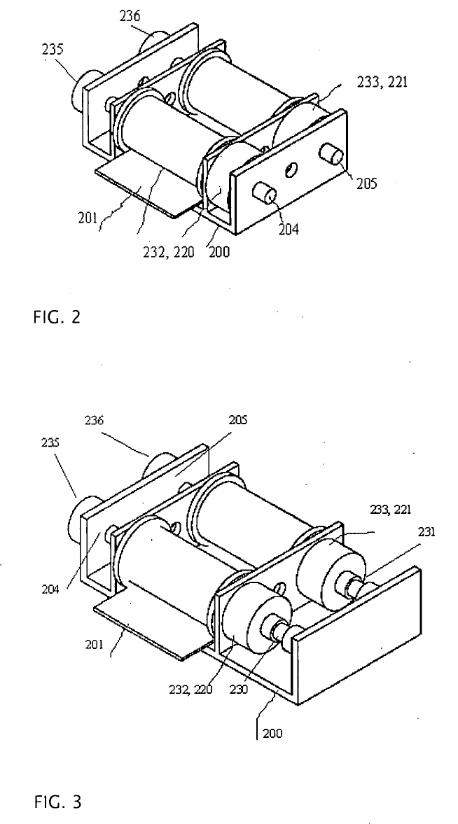

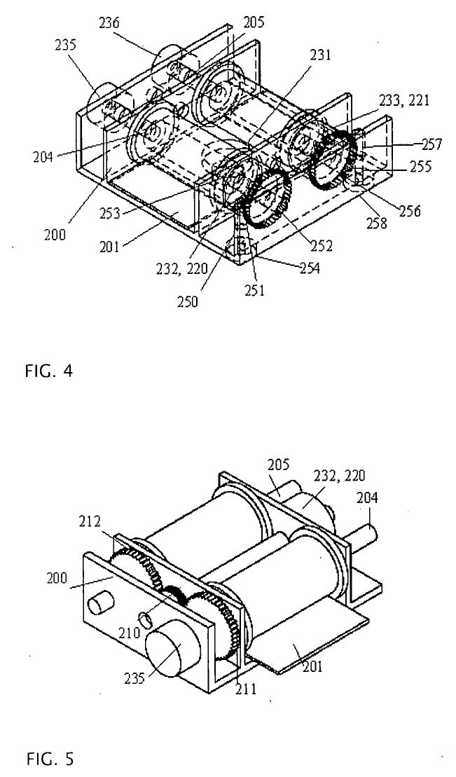

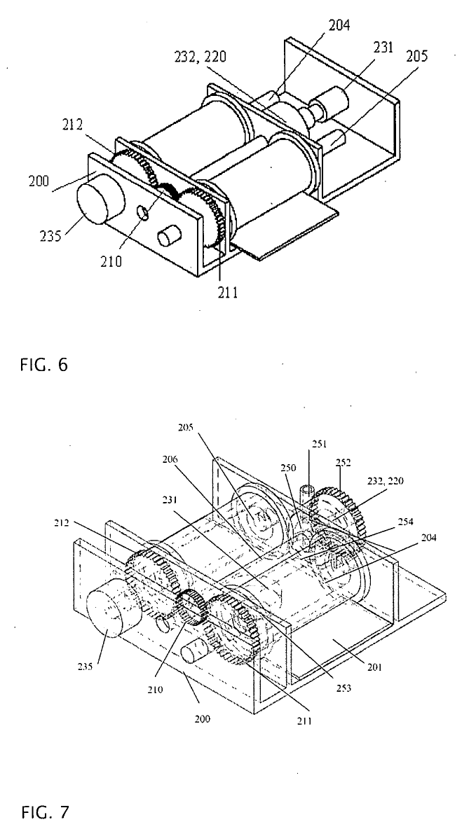

Referring to FIG. 2, in the first embodiment of the invention, the housing 200 contains rolling rods 204 and 205, spiral springs 220 and 221, spiral spring housings 232 and 233, and signal generators 235 and 236. The rolling rods 204 and 205 are located inside the housing 200 by being vertically inserted to the housing on the right and left hand sides, respectively. The belt 201 is rolled onto the rolling rods 204 and 205 such that it can be extended from both sides of the housing 200. The rolling rods 204 and 205 are generally free to rotate within the housing 200 about the vertical axis. The spiral springs 220 and 221 are connected to the rolling rods 204 and 205 such that any rotation of the rolling rods is passed onto the spiral springs to cause the spiral springs to compress (wind) or decompress (unwind) depending on a direction of rotation. Specifically, when the belt 201 coiled about the rolling rods 204 and 205 is being extended outwardly from the housing 200, the corresponding rotation of the rolling rods causes the spiral springs 220 and 221 to compress and to apply a torque onto the rolling rods in a direction opposite to the direction of rotation. The magnitude of torque applied onto the rolling rods 204 and 205 by the spiral springs 220 and 221 increases as the belt 201 coiled about the rolling rods is further expanded because the continuing rotation causes the spiral springs to become more and more wound up. If, the belt 201 is expanded outside the housing 200, but is not secured in that position, the force applied by the spiral springs 220 and 221 onto the rolling rods 204 and 205 about which the belt is coiled will cause the rolling rods to rotate in a direction opposite to the direction of expansion and will cause the belt to be retracted into the housing by being recoiled around the rolling rods. When attached about the body of the user 102, the user's respiratory movements will cause corresponding expansion and contraction of the belt 201, rotation of the rolling members 204 and 205, and compression and decompression of the spiral springs 220 and 221. More specifically, when the user 102 breathes in, the belt 201 is expanded and the torque applied by the spiral springs 220 and 221 onto the rolling rods 204 and 205 increases. When the user 102 breathes out, the diameter of the cross section of the user's body about which the belt 201 is attached decreases and the belt is partially retracted into the housing 200 to compensate for the decrease in diameter. This occurs automatically as the torque applied onto the rolling rods 204 and 205 by the spiral springs 220 and 221 causes the rolling rods to rotate and recoil slack portion of the belt 201 about them. One skilled in the art will appreciate and recognize that the tension of the spiral spring 220 and 221 is chosen such that the torque applied by the spiral springs onto the rolling rods 205 and 205, consequently causing the belt 201 to be tightly stretched around the body of the user 102 will not cause the user any discomfort or inconvenience but will merely be sufficient to hold the respiration monitor 100 in place and to precisely monitor the user's respiration. Furthermore, in the embodiments of the invention described in FIG. 3 and FIG. 4, FIG. 6 and FIG. 7, the user is able to control the force the belt applies to the users torso by being able to rotate the spiral spring housings.

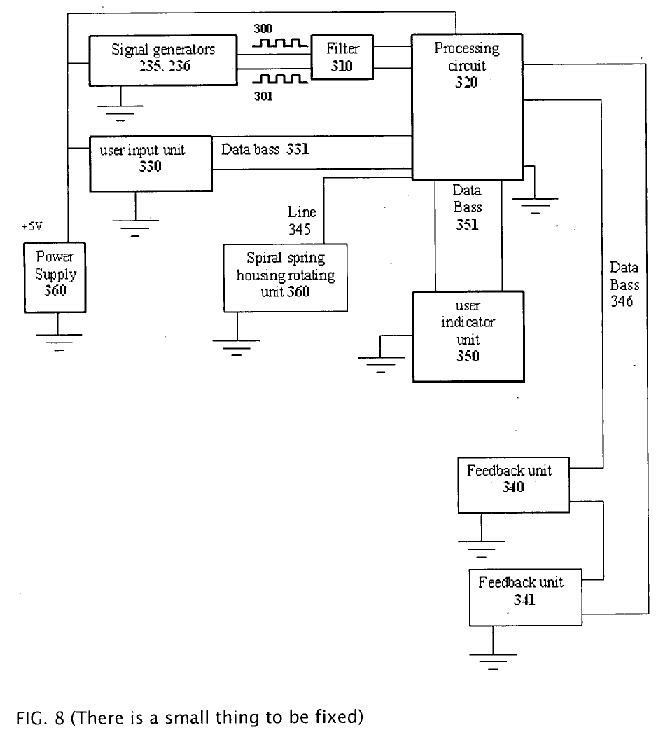

Still referring to FIG. 2, the signal generators 235 and 236 are connected to the rolling rods 204 and 205. The signal generators 235 and 236 generate a signal corresponding to the rotary movements of rolling rods 204 and 205. In the embodiments of the invention described herein, the signal generators 235 and 236 are rotary encoders, well known in the art, which generate square wave signals indicating angular changes of the rolling rods 204 and 205 resulting from the expansion and contraction of the belt 201 due to the respiratory movements of the user 102 as described above. One skilled in the art will appreciate and recognize that other types of signal generators may be used depending on the requirements of a specific application. The signal is received by processing circuit 320 described in FIG. 8, after being filtered by filter 310. The processing circuit performs a real time evaluation of the data stream signal from the signal generator based on the criteria and parameters setup by the user that will determine the given feedback.

Referring now to FIG. 3, the second embodiment of the invention is shown. In addition to the components described above in relation to the first embodiment of the invention, the spiral springs 220 and 221 are situated in a spring housing 232 and 233 that is not affixed to housing 200, rather it is connected directly to motor 230 and 231. By rotating the housing we are able to control the amount of force applied on the belt as it expands.

Referring now to FIG. 4, the third embodiment of the invention is shown. In addition to the components described above in relation to the first embodiment of the invention, the spiral springs 220 and 221 are situated in a spring housing 232 and 233 that is not affixed to housing 200, rather it is connected indirectly to motor 231 with a worm gear reduction. In this configuration, spiral spring housings 232 and 233 can be rotated in relation to the axis of spiral springs 220 and 221. Worm shafts 250 and 255 are situated on housing 200 perpendicular to the rotating axis of rolling rods 204 and 205 and rotates in relation to housing 200. Worms 251 and 257 are inserted into worm shafts 250 and 255, respectively, and they rotate with worm shafts 250 and 255. Worm gears 252 and 256 are affixed to spiral spring housings 232 and 233. Motor 231 is affixed to housing 200. Pulley 253 is affixed to motor 231. Pulleys 254 and 258 are affixed to worm shafts 250 and 255. Belt 259 (not shown) is situated on pulleys 253, 254 and 258 and transfers rotation of motor 231 to worm shafts 250 and 255. This configuration allows controlling the torque applied by spiral springs 220 and 221 on rolling rods 204 and 205, with the additional benefit of a self locking mechanism of a worm drive configuration.

In the embodiments described in FIG. 5 through FIG. 7, gear members 211 and 212 are fitted to the rolling rods 204 and 205, respectively. A rolling member 206 is free to rotate within housing 200. A gear member 210 is fitted to rolling member 206 such that the rolling movement of rolling rods 204 and 205 is transferred to rolling member 206. The spiral spring 220 is connected to the rolling member 206 such that any rotation of the rolling member is passed onto the spiral spring to cause the spiral spring to compress (wind) or decompress (unwind) depending on a direction of rotation as described above. Spiral spring 220 is situated in spiral spring housing 232. Signal generator 235 is connected to rolling rod 204. The signal generator generate signal corresponding to the rotary movements of rolling rod 204. In the embodiment of the invention described in FIG. 5, spiral spring housing 232 is affixed to housing 200 similar to the embodiment described in FIG. 2. In the embodiment of the invention described in FIG. 6, spiral spring housing 232 is connected directly to motor 231, similar to the embodiment described in FIG. 3. In the embodiment described in FIG. 7, spiral spring housing 232 is connected indirectly to motor 231 with a worm gear reduction, similar to the embodiment described in FIG. 4.

FIG. 8 describes the electronic circuitry related to the embodiments describe in FIG. 2 through FIG. 7. Signals 300 and 301 generated by signal generators 235 and 236 are transmitted through filter 310 to filter noise indicated by high frequencies. Processing circuit 320 receives the signals generated by signal generators 235 and 236 and filtered by a filter 310. Processing circuit 320 also receives user selected variables from user input unit 330 by data bass 331. Feedback units 340, 341 are connected to the processing circuit by data bass 346 and are activated or deactivated by the processing circuit

User indicator unit 350 is connected to processing circuit 320 by data bass 351 and it displays to the user active operation mode and the user selected parameters.

Spiral spring housing rotating unit 360 is related to the embodiment described in FIG. 3, FIG. 4, FIG. 6, and FIG. 7. It includes motors 230 and 231 connected to spiral spring housings 232 and 233. Spiral spring housing rotating unit 360 is connected to processing circuit 320 with line 345

We will assign the rotational angle (beta1) as the angular position of rolling rod 204 with respect to the starting position of rolling rod 204 when the belt is fully coiled within housing 200. We will assign the rotational angle (beta2) as the angular position of rolling rod 205 with respect to the starting position of rolling rod 205 when the belt is fully coiled within housing 200. As the user 102 breathes with the respiration monitor 100 is positioned in the diaphragm area, angles (beta1) and (beta2) change in correlation to the degree of expansion and contraction of the diaphragm of the user. In the embodiments of the invention described in FIG. 2 to FIG. 4, the total angle (alpha) is calculated as the sum of angle (beta1) and angle (beta2). In embodiments of the invention described in FIG. 5 to FIG. 7, the total angle (alpha) is calculated as angle (beta1) multiply by 2. This is due to the coupling of rolling rods 204 and 205 by gear members 210, 211 and 212.

The depicted embodiment utilizes a rotary encoder as signal generators 235 and 236. A rotary encoder generates an oscillating electrical signal having the form of a square-wave. The rotary encoder generates two square wave signals which differ from each other by a phase angle which is positive when the encoder rotates clockwise or negative when the encoder rotates counter-clockwise. Each square wave signal represents an angular movement of the encoder in constant angle. This angle is a function of the resolution of the encoder. Square wave signals generated by the incremental encoder that is used in the depicted embodiment as signal generator 235 are used by processing circuit 320 to determine the angular position of rolling rods 204 and 205.

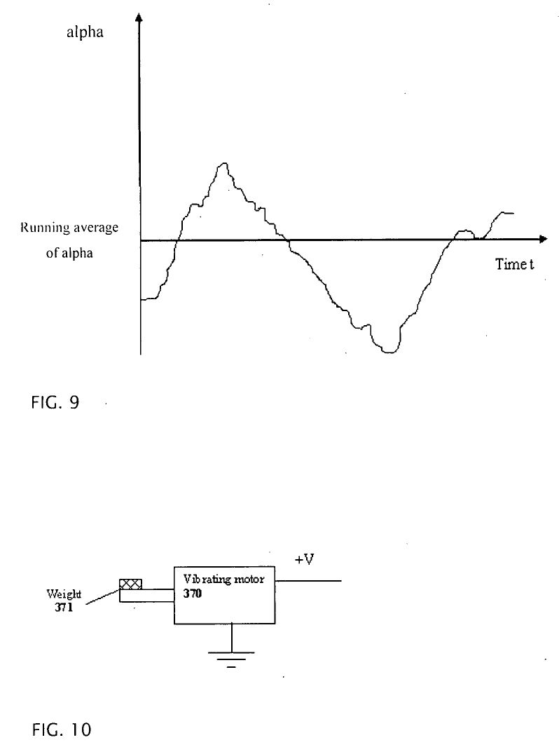

Processing circuit 320 receives the signals generated by signal generator 235 and 236, and creates a set of angular data with the corresponding internal clock times. This set of angular data represents respiratory patterns. An example of angular data and the corresponding internal clock times is described in FIG. 9

In one embodiment of the invention, described in FIG. 10, a vibrator motor 370 with a weight 371 is used as one of the feedback units 340 and 341 described in FIG. 8. The vibrator motor is used to transmit vibrations, also known as a vibration signal, for feedback to the user 102. The processing circuit 320 controls the pattern and duration of the vibrations.

In one embodiment of the invention, described in FIG. 8, feedback signal of gradually increasing force of the belt on the user's torso is utilized. The increasing force of the belt is controlled by the spiral spring housing rotating unit 360 describe in the above embodiments.

Other embodiments of the invention also utilize output devices as the feedback units 340 and 341 described in FIG. 8 that transmit auditory and/or visual feedback signals to the user 102.

The depicted embodiment uses light emitting diodes as the indicators for user indicator unit 350. The indicators are switched ON and OFF by processing circuit 320 according to the criteria that are discussed below. Devices other than light emitting diodes, such as LED displays, LCD displays, audio output devices or other devices known in the art to convey status and power information, are used by other embodiments of the invention.

Respiration rate and respiration depth are the two key respiration measurements performed by the respiration feedback monitor 100. Respiratory signals generated by signal generators 235 and 236 are received by processing circuit 320. Processing circuit 320 processes the signal and calculates the current absolute rotation angle (alpha). Current absolute rotation angle (alpha) and current internal clock time t are inserted into registers in a memory component in the processing circuit. These set of numbers that are generated from the angular movements of angular measurement device 330 are used to determine whether user 102 receives an active feedback previously defined by user 102.

We define initiation of a breath for a maximum rotational position cycle that follows a minimum rotational position cycle as the point where absolute rotation angle (alpha) becomes larger than the running average of the absolute rotation angle (alpha); we also define an initiation of a breath for a minimum rotational position cycle which follows a maximum rotational position cycle as the point where absolute rotation angle (alpha) becomes smaller than the running average of the absolute rotation angle (alpha).

The respiration feedback monitor 100 measures breathing rate by measuring the time between two consecutive breath initiation points (i.e., a respiration cycle). The respiration feedback monitor 100 measures respiration depth for a particular respiration cycle by calculating the extreme value of a maximum rotational position cycle, and the extreme value of a minimum rotational position cycle.

The depicted embodiment utilized a method to determine whether a feedback event should be activate or deactivate. The method includes sampling two sets of respiratory data. Each set of data includes angular data and time data representing the respiratory patterns, as described in FIG. 9. The first set of data includes the user's desired breathing pattern which is sampled during reset operational mode and will be assigned as reset-data. The second set of data includes the user's actual breathing pattern which is sampled during monitoring operational mode will be assigned as monitored-data. Feedback criterion is used to determine whether to activate or deactivate a feedback event. The feedback criteria uses the reset-data sampled during reset operational mode, and monitored-data sampled during monitoring operational mode. The feedback criteria also include user selected operational data determined by the user using input from unit 330, to determine the amount of feedbacks. Feedback criterion also considers the type of feedback selected by the user.

Reset operational mode is conducted to determine reset-data. In reset operational mode, set of respiratory data is sampled for a certain number of breathing cycles, for example, 20 cycles. The depicted embodiment determines whether the breathing is correct or incorrect according to the user's preferences. In this scope, a reset operational mode is conducted to allow the users to determine their desired breathing patterns. During reset operational mode, three sets of respiratory data containing breathing depth maximum values (Inhale), breathing depth minimum values (exhale), and breathing rate values (cycle times) are determined. Initiation of reset operational mode is selected by the user using user input unit 330. Reset operational mode is automatically deactivated after certain number of breathing cycles is conducted.

Monitoring operational mode is conducted to determine monitoring-data. In monitoring operational mode, set of respiratory data is sampled. During monitoring operational mode, three sets of respiratory data containing breathing depth maximum values (Inhale), breathing depth minimum values (exhale), and breathing rate values. (cycle times) are determined. Monitoring operational mode is selected by the user using input from unit 330.

Amount-of-feedback variable is determined by the user input from unit 330. In the depicted embodiment, the amount-of-feedback variable is determined by the user using a control knob. In other embodiments, other types of input devices, such as push buttons may also be utilized. Amount-of-feedback variable is used by the feedback criteria to determine how often the users want to be reminded with a feedback signal to improve their breathing.

The feedback criteria use the reset-data and monitoring-data determined by the reset operational mode and the monitoring operational mode respectively, as discussed above.

In the depicted embodiment of the invention, the criterion for whether to activate or deactivate a feedback event by testing the breathing depth patterns is determined statistically. In other embodiments of the invention, other criteria utilizing a comparison between the reset-data and monitoring-data may be utilized. In the depicted embodiment of the invention, the criterion for whether to activate or deactivate a feedback event is determined by conducting an f-test between the two sets of data, the reset-data and the monitoring-data. The reset-data and monitoring-data determined by the reset operational mode and the monitoring operational mode respectively, as discussed above. The f-test assesses whether the means of two groups are statistically different from each other. The f-test is used to determine whether the average maximum peak value of the monitoring-data is smaller than the average maximum peak value of the reset-data. The f-test is used to determine whether the average minimum peak value of the monitoring-data is larger than the average minimum peak value of the reset-data. In case one of these tests is true, the user fails to breathe properly according to breathing depth pattern. The feedback criterion counts the number of failures, and enters it into a variable we will assign as Amount-of-failures. It compares this variable to the value of Amount-of-feedback variable that is discussed above. In case the value of Amount-of-failures variable is smaller than the value of Amount-of-feedback variable, the feedback criterion increases its value by one, and erases the set of respiratory data we assigned as monitoring-data. Monitoring-data is sampled for a certain number of respiratory cycles, and the criterion is conducted again. In case the value of Amount-of-failures variable is equal or larger than the value of Amount-of-feedback variable, the feedback criterion increases is set to zero, and the set of respiratory data we assigned as monitoring-data is erased. The feedback event is triggered according to the selected user feedback type. Monitoring-data is sampled for a certain number of respiratory cycles, and the criterion is conducted again.

The depicted embodiment utilizes different type of user selected feedbacks. The user selects the feedback type with the user input from unit 330. In one of the embodiments of the invention, the selectable type of feedback involves a vibrator motor 370 with weight 371 as described in FIG. 10. The vibrator motor is activated by the processing circuit 320 to provide vibratory or tactile feedback to the user 102 when a feedback event is determined by feedback criteria described above. In the embodiments of the invention described in FIG. 3, FIG. 4, FIG. 6, and FIG. 7, the selectable type of feedback involves gradual increment of the force the belt applies on the user's torso. The gradual increment of the force is conducted by the activation of the spiral spring housing rotating unit 360 as discussed above. Another embodiment is an audio feedback provided by a digital music player that incorporates increase or decrease in volume into the device. The depicted embodiment includes other types of feedback and is not limited to what is described above.

The user using the embodiments described in FIG. 3, FIG. 4, FIG. 6, and FIG. 7 is able to determine the force of the belt 201 that is applied to the user's torso. Control of this force is conducted by the user utilizing input from unit 330. Processing circuit 320 receives the user selected parameters related to the force the belt applies to the user's torso, and correspondingly, activates spiral spring housing rotating unit 360 to increase or decrease the force the belt applies to the user's torso. This further increases the suability of the device because it allows the users to determine the force the belt applies to their torsos during different activities.

All of the above US patents and applications are incorporated by reference. While the depicted embodiment is used in training and rehabilitation for health conditions, other embodiments of the invention can similarly be used for monitoring and providing feedback related to other objectives, such as, for example, sports related activities, scientific research, voice training, or business office settings. Furthermore, aspects of the embodiments disclosed in the commonly assigned, co-pending Americal applications referenced above can be combined with aspects of the embodiments disclosed herein. For instance, aspects of the Portable Respiration Monitoring and Feedback System could be combined with aspects disclosed herein resulting in a feedback monitor for a user's muscle and respiration activities. As an alternative example, aspects of the Heart Rate Variability Feedback Monitor System could be combined with aspects disclosed herein resulting in a feedback monitor for a user's heart and respiration activities.

These and other changes can be made to the invention in light of the above detailed description. In general, in the above claims, the terms should not be construed to limit the invention to specific embodiments disclosed in the claims, but should be construed to include all wearable respiration feedback monitors that operate under the claims to provide a wearable system for monitoring and providing appropriate feedback related to respiration activity of the user, and to all feedback systems operating under one or more of the above methods. Accordingly, the invention is not limited by the disclosure, but instead its scope is to be determined entirely by the preceding.

We claim:

1. A portable respiration monitoring and feedback system comprising:
a housing, sized and configured to be worn by a user;
a belt coiled within the housing that extends from both sides of the housing and configured to girdle the user's abdomen or torso;
Rolling rod configured to rotate to coil and uncoil a belt that corresponds to the user's respiration;
Signal generator affixed to the housing configured to rotate with the rolling rod. The signal generator generates signals that correspond to the rotary movement of the rolling rod;
User input unit affixed to the housing configured to generate signals corresponding to user selected parameters;
User indicator unit affixed to the housing configured to display the user data related to respiratory and user selected parameters;
Processing circuit affixed to the housing configured to receive the signals generated by the signal generator and the signals generated by the user input unit, and to turn on a output device if the signal generator signals do not satisfy respiration feedback criteria under which the processing circuit operates. The processing circuit is further configured to generate signals related to respiratory and user selected parameters that are transmitted to the user indicator unit;
Output device affixed to the housing configured to transmit a signal perceptible by the user when activated;

2. The invention of claim 1 wherein:
Spiral spring is connected to the rolling rods and is configured such that any rotation of the rolling rod is passed onto the spiral spring to cause the spiral spring to compress (wind) or decompress (unwind) depending on a direction of rotation;
Spiral spring housing is configured to contain the spiral spring;

3. The invention of claim 1 wherein:
Gear members inserted to each of the rolling rods configured to transmit the rotation movement of one of the rolling rods to the other rolling rod;

4. The invention of claim 1 wherein:
Rolling member configured to rotate within the housing;
Gear members configured to transmit rotational movements of the rolling rods to the rolling member;
Spiral spring connected to the rolling member and is configured such that any rotation of the rolling member is passed onto the spiral spring to cause the spiral spring to compress (wind) or decompress (unwind) depending on a direction of rotation;
Spiral spring housing configured to contain the spiral spring;

5. The invention of claim 2 where:
The spiral spring housing is affixed to housing;

6. The invention of claim 2 where:
Motor coupled to the spring housing such that the spring housing can be rotated by the motor in the direction of the spring axis;

7. The invention of claim 2 where:
Worm gear attached to the gear housing;
Worm shaft perpendicular to the gear housing and inserted into holes in the housing such that it can rotate in the perpendicular direction of the axis of the gear housing;
Worm inserted and affixed to the worm shaft;
Pulley inserted to the worm shaft;
Motor affixed to the housing;
Pulley inserted to the motor shaft;
Belt configured to connect the pulleys of the worm shaft and the motor shaft such as the rotational movement of the motor is transferred to the worm shaft;

8. The invention of claim 4 where:
The spiral spring housing is affixed to housing;

9. The invention of claim 4 where:
Motor coupled to the spring housing such that the spring housing can be rotated by the motor in the direction of the spring axis;

10. The invention of claim 4 where:
Worm gear attached to the gear housing;
Worm shaft perpendicular to the gear housing and inserted into holes in the housing such that it can rotate in the perpendicular direction of the axis of the gear housing;
Worm inserted and affixed to the worm shaft;
Pulley inserted to the worm shaft;
Motor affixed to the housing;
Pulley inserted to the motor shaft;

11. A method compromising:
a Reset procedure where in the reset procedure the users determine their desired respiratory pattern;
a Monitoring procedure where in the monitoring procedure the users respiration in monitored and tested according to feedback criteria to determine whether a feedback event should be activated or deactivated

12. The invention in claim 10 where:
a feedback criterion where the sets of respiratory data sampled during the monitoring and reset procedures are tested whether they have F-distribution;
